(19) United States
(12) Patent Application Publication
(10) Pub. No.: US 2008/0139671 A1
(43) Pub. Date: Jun. 12, 2008

(54) METHOD AND MATERIAL FOR IN SITU CORNEAL STRUCTURAL AUGMENTATION

(75) Inventor: Satish Herekar, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: PriaVision, Inc., Menlo Park, CA (US)

(21) Appl. No.: 11/952,801

(22) Filed: Dec. 7, 2007

Related U.S. Application Data
(60) Provisional application No. 60/869,048, filed on Dec. 7, 2006.

(51) Int. Cl. A61K 47/16; A61P 27/02

(52) U.S. Cl. 514/773

(57) ABSTRACT

A method and material for augmenting the shape and thickness of the cornea in situ includes applying a clear liquid collagen mixed with a customized crosslinker onto the augmentation surface or in a cavity (with or without a mold) and exposing the mixture to UVA radiation in vivo. Application of UVA at varying dosages demonstrate progressive optically clear gelation and biomechanical adherence properties, and in vitro optical properties (RI), mechanical suture strength and rheometric parameters are comparable to native corneal stromal tissue. Photochemical corneal collagen augmentation according to the invention makes it suitable to reconstruct and strengthen diseased and damaged eyes, ulcerated corneas, as well as provide a substrate for refractive onlay/inlay procedures and lamellar transplantation.

### CROSS-REFERENCES TO RELATED APPLICATIONS

NOT APPLICABLE

### STATEMENT AS TO RIGHTS TO INVENTIONS MADE UNDER

### FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### BACKGROUND OF THE INVENTION

The present invention relates generally to biomedical techniques. More particularly, the invention relates to a method and materials for augmenting corneal, scleral, and retinal tissue and for treating, such as by repairing and reshaping, ocular tissues and eventually for refractive surgery.

Corneal and other ocular structural weakness, such as scleral structural weaknesses, can have several origins, including genetic, iatrogenic, accidents and shortcoming of desired surgical correction. Furthermore, ulcerations, melts, and the like, may require localized repair. Refractive corrections refer to either corneal reshaping surgery or addition of prosthetics (inlays/onlays/cavity augmentations) or some combination thereof. Localized repair is currently performed by lamellar surgery and requires precise in situ “fitting” of biocompatible host and donor tissues and maintaining smooth interfaces and biocompatibility thereafter, all of which are not insignificant issues. Laser-based surface shaving surgery complications are well publicized and may be easily referenced in current literature. Suturing has its own set of difficulties and shortcomings, as does tissue gluing.

It is long known that collagen exposed to riboflavin, also known as vitamin B2, in the presence of ultraviolet light produces cross-linking, which is useful as a cell scaffold for rebuilding cartilaginous defects. It is also known that corneal tissue can be stiffened by cross-linking by UVA irradiation in the presence of riboflavin eyedrops. However, problems with riboflavin-mediated cross-linking in collagen include shrinkage and increased opacity. (Representative citations herein are not necessarily prior art.) In the ocular domain, as published since the filing of the priority application upon which this invention is based, work has been reported on techniques for corneal cross-linking by photopolymerization of stromal fibers in the presence of riboflavin by irradiation with ultraviolet light. See Cosimo Mazzotta PhD, Angelo Balestrazzi PhD, Stefano Baiocchi PhD, Claudio Traversi MD PhD, Aldo Caporossi MD (2007), “Stromal haze after combined riboflavin-UVA corneal collagen cross-linking in keratoconus: in vivo confocal microscopic evaluation,” Clinical & Experimental Opthalmology, Volume 35 (6), pp. 580-582, (August 2007). (doi:10.1111/j.1442-9071.2007.01536.x). No separate application of augmentation materials or suggestions of mixtures of photoreactive augmentation materials was reported in that study. Moreover, the researcher reported negative results: The therapy caused stromal haze after the cross-linking treatment.

Others have reported on the results of collagen cross-linking induced by riboflavin exposed to UVA. Wollensak reported on collagen cross-linking induced by riboflavin UVA and involving injection of a polymeric composition forming a gel into the eye in The Journal of Cataract & Refractive Surgery, Vol. 30 (3), pp. 689-695 (March 2004). Augmentation by onlay in particular was not addressed.

Therefore, there remains in the art a need for a method for effective augmentation therapy, and well as the formulation of materials that can be used in effective augmentation therapy, for ocular applications.

### SUMMARY OF THE INVENTION

According to the invention, a method and material for augmenting the shape and thickness of ocular tissues, in particular the cornea, in situ are disclosed. The method includes applying a clear liquid collagen mixed with a customized cross-linker, either as a layer or as molding material, depending upon customized thickness/shape properties, and exposing the mixture to ultraviolet radiation, typically UVA, in vivo, for a period corresponding to the thickness of the stratum, and typically less than five minutes. Application of UVA at varying dosages yields progressive optically clear gelation and biomechanical adherence properties, and the in-vitro optical properties, mechanical suture strength and rheometric characteristics are comparable to native corneal stromal tissue. Photochemical-based corneal collagen augmentation according to the invention makes it suitable for clinical use to reconstruct and strengthen diseased and ulcerated corneas, as well as provide a substrate for refractive augmentation procedures and lamellar transplantation, in particular as a suitable therapy for corneal augmentation.

The invention will be better understood by reference to the following detailed description.

### BRIEF DESCRIPTION OF THE DRAWINGS

NOT APPLICABLE

### DETAILED DESCRIPTION OF THE INVENTION

A suitable photochemical-based ocular tissue and corneal augmentation material according to the invention comprises clear liquid collagen of bovine, porcine and recombinant human origin at concentration levels of about 0.5% to about 7% weight per volume, and preferably between 1% and 5% wpv, at a pH between 6.5 and 7.0, mixed with a customized, non-toxic, water soluble cross-linker (XL, see below) having the active ingredient riboflavin in dilutions of around at least 1:500 to about 50:500 and preferably between 1:100 and 5:100 (XL:Collagen). A method according to the invention involves applying the fluid mixture (a formable gel) to ocular tissue to a preselected profile and thickness and irradiating the formable gel with ultraviolet energy that is reactive with the cross-linker in the mixture, in particular by irradiating with directed or focused or coherent (laser) ultra-violent light and more particularly Ultraviolet Type A (UVA) radiation at varying dosages, such as ˜370 nm UVA light at 6 mW/cm² to 30 mW/cm² for varying periods, such as 1 minute to about 5 minutes to 15 minutes and as long as about 30 minutes. As a result, the material polymerizes into an augmenting stratum that adheres to the treated host substrate. Tests of the material in representative trials resulted in establishing suitable parameters of concentration, wavelength and exposure intensity and exposure time for collagen/riboflavin mixtures for corneal augmentation over a range of thicknesses. The following is a table showing what are believed to be baseline parameters for corneal augmentation of various thicknesses:

Optimal Parameters for Corneal Augmentation

| | Collagen | XL:Coll | | |
|---|---|---|---|---|
| Thickness | Conc.(w/v) | Dilution | UVA fluence | Exp Time |
| 100 μm | 1% | 1:500 | 12 mW/cm² | 3 min. |
| | | | (6-30) | |
| 200 μm | 3% | 1:100 | 12 mW/cm² | 4 min. |
| | | | (6-30) | |
| 300 μm | 5% | 5:100 | 12 mW/cm² | 5 min. |
| | | | (6-30) | |

In ranges of collagen concentrations of 0.5% to 7% weight per volume collagen and cross-linker to collagen dilution of 1:500 to 1:100, there is a tradeoff between mechanical strength vs. optical clarity and transparency for thin augmentation layers. Thus, the table shows nominal effective values, subject to variances within a reasonable range. Examination of the boundaries of the variances demonstrate that the values are valid over a range of an estimated three standard deviations from the stated optimal parameters without loss of cross-linking effectiveness. Mechanical strength for example is increased by increasing collagen concentration. Increasing fluence time beyond 10 minutes does not lead to further improvement in properties. Procedures and results are listed in Appendix A, fully incorporated herein. The results were optically clear and progressive gelation reaching a stable state after the end of exposure. There is little further gelation after about one hour, since the riboflavin cross-linker no longer produces reactive species. The resultant object binds with the underlying substrate, typically living tissue, and it can be subjected to other procedures, such as excimer laser-based ablation, femto-second pulsed ablation, or other conventional surgical procedures. The photochemical gelation procedure was tested for biomechanical adherence properties in vitro and on moldings of tissue or collagen in varying thicknesses (100 μm, 200 μm, 400 μm, and 100 μm+100 μm—a multilayer cross-linking laminate).

Preliminary in vitro lab results (following initial optimization of the cross-linking procedure) of optical properties (RI), mechanical suture strength and rheometric measurements compared to native corneal stromal tissue were found to be equivalent or better.

In situ collagen corneal molding using UVA and riboflavin combinations are able to produce a clear adherent layer of transparent collagen on top of corneas, as well as in Petri dishes and contact lenses.

The procedure of photochemical augmentation has biocompatibility, as well as optical, biomechanical and adhesive properties, suitable for human therapy with the potential to reconstruct and strengthen diseased and ulcerated corneas, scleral and other ocular tissues, to use in strabismus of ocular muscle or tendon repair, as well as provide a substrate for refractive onlay/inlay procedures, cavity augmentation and lamellar transplantation.

The procedure may be implemented with a mold formed for example by a contact lens, or it may be used without a mold, such as by spray application of photo-chemically cross-linkable collagen mixtures.

Various factors which represent tradeoffs may be manipulated to optimize for desired results. These include collagen concentration, which is correlated with mechanical and optical properties, acidity, which is correlated with translucency and biocompatibility, cross-linker concentration and UVA exposure time, related to setting of the shape of collagen, which is correlated with ease of manipulation and potential for radiation damage to the tissue, and thickness of applied layer per exposure, which is a material consideration. The procedure herein disclosed contemplates single or multi-regional photo-initiating exposure, as well as exposure of large contiguous areas.

The invention has been explained with reference to specific embodiments. Other embodiments will be evident to those of ordinary skill in the art. It is therefore not intended that the invention be limited, except as indicated by the appended claims.

APPENDIX A

| | | | |
|---|---|---|---|
| trial 1 | chill on ice for 10 mins water, collagen xl | 30 mg/ml final | 66 mg/ml init |
| | syringe 1 ml collagen soln | BD 3 mL luer lock tip | |
| | 1:100 XL | syringe | |
| | syringe mix 500 uL water | | |
| | remove bubbles syringe mix | | |
| | add 400 uL water syringe mix | | |
| | remove bubbles syringe mix | | |
| | HCL 100 uL add | | |
| | 2 syringe mix to form clear soln ~2 mL | | |
| | 20 uL XL soln mixed with 2 ml | | |
| | ph measures 2 | | |
| | UVA 10 mw/cm2exposure 2 mins not still gelled | | |
| | UVA Oven varies hi to lo 20 mW/cm2 to 10 mW/cm2 | | |
| | UVA exposure 3 mins not gelled, 10 mW/cm2 | | |
| trial 2 | syringe 1 ml collagen soln | | |
| | 1:10 XL | | |
| | syringe mix 500 uL water | | |
| | remove bubbles syringe mix | | |
| | add 400 uL water syringe mix | | |
| | remove bubbles syringe mix | | |
| | HCL 100 uL add | | |
| | 2 syringe mix to form clear soln ~2 mL | | |
| | 200 uL XL soln mixed with 2 ml | | |
| | ph measures 2 | | |
| | UVA 10 mw/cm2 exposure 5 mins gelled but temp too high | | |
| trial 3 | syringe 1 ml collagen soln | | |
| | 5:100 XL | | |
| | syringe mix 500 uL water | | |
| | remove bubbles syringe mix | | |
| | add 400 uL water syringe mix | | |
| | remove bubbles syringe mix | | |
| | HCL 100 uL add | | |
| | 2 syringe mix to form clear soln ~2 mL | | |
| | add 50 uL 1N NaOH, syringe mix | | |
| | 100 uL XL soln mixed with 2 ml | | |
| | ph measures 3.6 | | |
| | UVA 10 mw/cm2exposure 3 mins gelled | | |
| trial 4 | syringe 1 ml collagen soln | | |
| | 5:100 XL | | |
| | syringe mix 500 uL water | | |
| | remove bubbles syringe mix | | |
| | add 400 uL water syringe mix | | |
| | remove bubbles syringe mix | | |
| | HCL 100 uL add | | |
| | 2 syringe mix to form clear soln ~2 mL | | |
| | add 50 uL 1N NaOH, syringe mix | | |
| | 100 uL XL soln mixed with 2 ml | | |
| | ph measures 5.2 | | |
| | UVA 11 mw/cm2 exposure 3 mins gelled | | |
| trial 5 | syringe 1 ml collagen soln | | |
| | 5:100 XL | | |
| | syringe mix 500 uL water | | |
| | remove bubbles syringe mix | | |
| | add 400 uL water syringe mix | | |
| | remove bubbles syringe mix | | |
| | HCL 100 uL add | | |
| | 2 syringe mix to form clear soln ~2 mL | | |
| | add 50 uL 1N NaOH, syringe mix | | |
| | 100 uL XL soln mixed with 2 ml | | |
| | ph measures 5.5 | | |
| | paraffin wax paper and Petri dish collagen chip | | |
| | UVA 11 mw/cm2 exposure 3 mins gelled OPTICAL CLARITY GOOD | | |
| trial 6 | syringe 1 ml collagen 50 mg/ml soln | | |
| | 1:10 XL | | |
| | syringe mix 30 uL water | | |
| | remove bubbles centrifuge 5 mins 10k rpm | | |
| | HCL 160 uL add | | |
| | 2 syringe mix to form clear soln ~1.13 mL | | |
| | add 60 uL 1N NaOH, syringe mix | | |
| | 130 uL XL soln mixed with 1.12 ml | | |
| | ph measures 6.1 | | |
| | paraffin wax paper and Petri dish collagen chip | | |
| | UVA 11 mw/cm2 exposure 3 mins no gel | | |
| trial 7 | DOUBLE LAYERED XL PETRI DISH | | |
| | syringe 1 ml collagen soln | | |
| | 5:100 XL | | |
| | syringe mix 500 uL water | | |
| | remove bubbles syringe mix | | |
| | add 400 uL water syringe mix | | |
| | remove bubbles syringe mix | | |
| | HCL 100 uL add | | |
| | 2 syringe mix to form clear soln ~2 mL | | |
| | add 50 uL 1N NaOH, syringe mix | | |
| | 100 uL XL soln mixed with 2 ml | | |
| | ph measures 5.5 | | |
| | paraffin wax paper and Petri dish collagen chip | | |
| | UVA 11 mw/cm2 exposure 3 mins gelled | | |
| trial 8 | DOUBLE LAYERED XL CL | | |
| | syringe 1 ml collagen soln | | |
| | 5:100 XL | | |
| | syringe mix 500 uL water | | |
| | remove bubbles syringe mix | | |
| | add 400 uL water syringe mix | | |
| | remove bubbles syringe mix | | |
| | HCL 100 uL add | | |
| | 2 syringe mix to form clear soln ~2 mL | | |
| | add 50 uL 1N NaOH, syringe mix | | |
| | 100 uL XL soln mixed with 2 ml | | |
| | ph measures 5.5 | | |
| | paraffin wax paper and Petri dish collagen chip | | |
| | UVA 11 mw/cm2 exposure 3 mins gelled | | |
| trial 9 | initial 98 mg/ml, target 50 mg/ml | vol = 1.96 ml | vol of |
| | syringe 1 ml collagen soln | | water = 660 ul |
| | 1:10 XL | | |
| | syringe mix vigorously 500 uL water | | |
| | remove bubbles syringe mix | | |
| | add 160 uL water syringe mix | | |
| | remove bubbles syringe mix | | |
| | HCL 100 uL add | | |
| | 2 syringe mix to form clear soln ~2 mL | | |
| | 200 uL XL soln mixed with 2 ml | | |
| | ph measures 5.5 | | |
| | paraffin wax paper and Petri dish collagen chip | | |
| | UVA 11 mw/cm2 exposure 3 mins gelled OPTICAL CLARITY GOOD | | |
| trial | initial 98 mg/ml, target 50 mg/ml | | |
| 10 | syringe 1 ml collagen soln | | |
| | 5:100 XL | | |
| | syringe mix vigorously 500 uL water | | |
| | remove bubbles syringe mix | | |
| | add 160 uL water syringe mix | | |
| | remove bubbles syringe mix | | |
| | HCL 100 uL add | | |
| | 2 syringe mix to form clear soln ~2 mL | | |
| | add 50 uL 1N NaOH, syringe mix | | |
| | 100 uL XL soln mixed with 2 ml | | |
| | ph measures 5.5 | | |
| | add 50 uL water | | |
| | paraffin wax paper and Petri dish collagen chip | | |
| | UVA 11 mw/cm2 exposure 2, 3, 5 mins gelled OPTICAL CLARITY GOOD | | |
| trial | initial 98 mg/ml, target 10 mg/ml | Vol = 4.9 ml | vol of |
| 11 | syringe 0.5 ml collagen soln | | water = 4150 |
| | 5:100 XL | | |
| | syringe mix with 2000 ul water | | |
| | add 2150 ul water and HCL 50 ul to get collagen solution | | |
| | add 25 ul 1N NaOH, 200 ul XL soln mixed to form clear soln ~5 mL | | |
| | UVA 11 mw/cm2 exposure 2, 3, 5 mins | | |

What is claimed is:

1. A method for augmenting ocular tissue comprising:
mixing clear liquid collagen selected from one of bovine, porcine and recombinant human origin with a small percentage of cross-linker having as the active ingredient riboflavin to obtain a mixture;
applying the mixture to a living eye tissue; and
exposing the mixture in situ to Ultraviolet A (UVA) radiation at dosages between 6 mW/cm² and 30 mW/cm² for periods between 1 minute to 30 minutes to form a layer of tissue augmentation of selected thickness.

2. The method according to claim 1 wherein the living eye tissue is corneal tissue and said tissue augmentation is substantially clear.

3. The method of claim 1 wherein optimal and range parameters are substantially as follows:

| Approx. | Collagen | XL:Coll. | | |
|---|---|---|---|---|
| Thickness | Conc.(w/v) | Dilution | UVA fluence | Exp. Time |
| 100 μm | 5% (0.5-7) | 1:500 | 12 mW/cm² | 3 min. |
| | | (1-50:500) | (6-30) | (1-5) |
| 200 μm | 5% (0.5-7) | 1:100 | 12 mW/cm² | 4 min. |
| | | (1-50:500) | (6-30) | (1-15) |
| 300 μm | 5% (0.5-7) | 5:100 | 12 mW/cm² | 5 min. |
| | | (5-50:500) | (6-30) | (1-30) |

4. A material for promoting augmentation of ocular tissue comprising:
an ultraviolet radiation-reactive cross-linker mixed into liquid collagen.

5. The material according to claim 4 wherein said ultraviolet radiation-reactive cross-linker is reactive to UVA.

6. The material according to claim 5 wherein said reactive to UVA cross-linker is riboflavin.

7. The material according to claim 6 wherein said liquid collagen is selected from bovine, porcine and recombinant human collagen.

8. A method for augmenting corneal and ocular tissue comprising the steps of:
applying a fluid mixture of liquid collagen having an unreacted ultraviolet radiation-reactive cross-linker to stromal tissue; and
irradiating in vivo said fluid mixture upon said corneal tissue with optical radiation of an effective wavelength and in sufficient dosage and duration sufficient to cause binding to said stromal tissue and gelation of a preselected rigidity in said fluid mixture.

9. The method according to claim 8 wherein said fluid mixture is clear and said ocular tissue is corneal tissue.

10. A method for augmenting ocular tissue including corneal tissue comprising the steps of:
applying a fluid mixture of liquid collagen having therein an unreacted ultraviolet radiation-reactive cross-linker to a mold;
irradiating said fluid mixture in vitro upon said mold with optical radiation of an effective wavelength and in sufficient dosage and duration to cause gelation in a preselected rigidity in said fluid mixture; and thereafter
causing the binding said fluid mixture to ocular tissue.

11. The method according to claim 10 wherein said fluid mixture is clear and said ocular tissue is corneal tissue.
